(19) United States
(12) Patent Application Publication
(10) Pub. No.: US 2010/0028467 A1
(43) Pub. Date: Feb. 4, 2010

(54) Composition Comprising Ginsenosides Isolated From The Extract Of Processed Ginseng For Preventing And Treating Thrombotic Disease

(75) Inventors: Bok Deuk KIM, Seoul (KR); Jeong Hill PARK, Seoul (KR); Hye Sook YUN-CHOI, Seoul (KR); Jin Gyun LEE, Seoul (KR); Yong Yook LEE, Seoul (KR)

Correspondence Address: KIRK HAHN, 14431 HOLT AVE, SANTA ANA, CA, 92705, US

(73) Assignees: Ginseng Science, Inc., Seoul (KR); Seoul National University R&DB Foundation, Seoul (KR)

(21) Appl. No.: 12/512,875

(22) Filed: Jul. 30, 2009

(30) Foreign Application Priority Data
Jul. 31, 2008 (KR) 10-2008-0075260

(51) Int. Cl. A61K 36/258; A61K 31/704

(52) U.S. Cl. 424/728; 514/26

(57) ABSTRACT

The present invention relates to compositions containing an extract of processed Panax genus plant and ginsenosides isolated therefrom for treat thrombotic diseases in human or mammal, the method for treating said diseases using thereby and the use thereof.

### BACKGROUND OF THE INVENTION

1. Technical Field

The present invention relates to compositions containing an extract of processed Panax genus plant and saponin compounds isolated therefrom for treat thrombotic disease in human or mammal, the method for treating said diseases using thereby and the use thereof. More particularly, the present invention relates to a use of an extract of processed ginseng and saponin compounds isolated therefrom with enhanced pharmacological effects due to heat-treatment of a Panax genus plants so as to make a ratio of ginsenoside (Rg3+Rg5+Rk1) to (Rb1+Rb2+Rc+Rd) of over 1.0.

2. Background Art

To prevent thrombus formation and to remove already formed thrombus effectively is crucial factor in treating vascular disease such as cardiac failure, hypertension, hyperlipemia, arrhythmia, and thrombosis since thrombus plays important roles in the progress of cardiovascular disease

The drugs for the prevention and treatment of thrombotic disease can be classified into three categories, i.e., a thrombolytic agent used for the purpose of first aid drug by dissociating already formed fibrin, a main component of thrombus; an anti-coagulant agent such as heparin, coumarin etc by blocking blood coagulation system resulting in inhibiting thrombus formation; and anti-platelet agent. However, those drugs have several adverse action such as hemorrhage at vascular damaged region, systemic hemorrhage etc and therefore are not suitable for long-term treatment

Several substances produced in human body act on the formation of thrombosis. Especially, the conventional mechanism of platelet aggregation inhibition is by antagonizing several substances such as arachidonic acid, collagen, thromboxane A2 etc. Therefore, there have been needed to develop new agent to inhibit thrombotic diseases by antagonizing those substances till now.

It is known that there are many genus of Panax genus plants belonged to Araliaceae, for example, Panax ginseng distributed or cultivated in far-eastern Asia region, Panax quinquefolia in America and Canada, Panax notoginseng in China, Panax trifolia in eastern region of north America, Panax japonica in Japan, China and Nepal, Panax pseudoginseng in Nepal, Panax vietnamensis in Vietnam, Panax elegatior, Panax wangianus and Panax bipinratifidus etc.

It has been known that the main constituent of Panax genus plant is dammarane-skeleton type saponins, such as ginsenosides Rb₁, Rb₂, Rc, Rd, Rg1 and Re etc. The biological activities of them are different from each other in accordance with their chemical structures and ginseng has been reported to be effective in fatigue recovery or vigor reinforcement from long years ago.

Recently, there have been several attempts to strengthen pharmacological effects of ginseng by modifying the method of ginseng processing for example, Park et al developed new methods for preparing a processed ginseng under specific high temperature and high pressure as disclosed in Korean Patent Registration No. 192678 and U.S. Pat. No. 5,776,460, which changes main ginseng saponins such as ginsenosides Rb1, Rb2, Rc and Rd, into new saponin metabolites such as ginsenosides Rg3, Rg5 and Rk1 showing new and more potent pharmacological effects, for examples, anti-oxidative activity, anti-cancer activity and alleviating activity of blood circulation etc (Kim W Y et al., J. Nat. Prod., 63(12), pp. 1702-1704). Ginsenosides are well known to undergo structural changes during heat processing. This new processed ginseng contains ginsenosides Rg₃, Rg₅, Rk₁, Rk₂, Rk₃, Rs₄, Rs₅, Rs₆ and Rs₇ which are produced by deglycosylation and dehydration at the position C₂₀ in dammarane backbone (Kwon S W, Han S B, Park I H, Kim J M, Park M K, Park J H (2001) Liquid chromatographic determination of less polar ginsenosides in processed ginseng. J Chromatogr A 921: 335-339; Park I H, Han S B, Kim J M, Piao L, Kwon S W, Kim N Y, Kang T L, Park M K, Park J H (2002a) Four new acetylated ginsenosides from processed ginseng (sun ginseng ). Arch Pharm Res 25: 837-841; Park I H, Kim N Y, Han S B, Kim J M, Kwon S W, Kim H J, Park M K, Park J H (2002b), Three new dammarane glycosides from heat processed ginseng. Arch Pharm Res 25: 428-43). These components, not usually found in white ginseng, are thought to be responsible for enhanced pharmacological effect of processed ginseng.

Accordingly, these new metabolites can be produced in the root, stem or leaf of any panax genus plants such as Panax ginseng, Panax quinquefolia, Panax notoginseng, Panax trifolia, Panax japonica, Panax pseudoginseng, Panax vietnamensis, Panax elegatior, Panax wangianus and Panax bipinratifidus which contains dammarane glycoside through the processing method of Park et al (Korean Patent Registration No. 192678 and U.S. Pat. No. 5,776,460).

However, there has been not reported or disclosed about the treating or preventing effect of the processed ginseng extract prepared by above-described methods and several ginsenosides isolated therefrom on thrombotic disease in any of above cited literatures, the disclosures of which are incorporated herein by reference.

Accordingly, the present inventors have discovered that the extract of processed ginseng product as to make a ratio of ginsenoside (Rg3+Rg5+Rk1) to (Rb1+Rb2+Rc+Rd) of over 1.0 and the ginsenosides isolated therefrom show potent inhibitory effect on platelet aggregation induced by AA, thromboxane A2 and collagen, and finally completed the present invention.

### SUMMARY OF THE INVENTION

The present invention also provides a use of extract of processed ginseng and ginsenosides isolated therefrom for the prevention or treatment of thrombotic disease. The present invention provides a pharmaceutical composition and a health food comprising extract of processed ginseng and ginsenosides isolated therefrom as an active ingredient in an effective amount to treat and prevent thrombotic disease.

The present invention also provides a method for treating or preventing of thrombotic disease in human or mammal comprising administrating to said mammal an effective amount of above extract of processed ginseng and ginsenosides isolated therefrom, together with a pharmaceutically acceptable carrier thereof.

### DISCLOSURE OF THE INVENTION

In accordance with the present invention, the present invention provides a use of extract of processed ginseng and the ginsenosides isolated therefrom for the preparation of medicament employed for preventing or treating thrombotic disease in human and mammals.

In accordance with the present invention, the present invention also provides a composition comprising an extract of processed ginseng and ginsenosides isolated therefrom as an active ingredient in an effective amount to treat and prevent thrombotic disease.

The present invention also provides a method for treating or preventing of thrombotic disease in human or mammal comprising administrating to said mammal an effective amount of an extract of processed ginseng and ginsenosides isolated therefrom, together with a pharmaceutically acceptable carrier thereof.

The term “an extract of processed ginseng ” disclosed herein comprises an extract of processed ginseng obtained by the steps; treating Panax genus plants with heat at the temperature ranging from 70 to 150° C. for the period ranging from 2 to 6, as an active ingredient in an amount effective to treat or prevent thrombotic disease, which can be prepared by the procedure disclosed in Korean Patent Registration No. 192678 and U.S. Pat. No. 5,776,460).

The term ginsenoside “Rg3” disclosed herein includes two isomers of ginsenoside (20-S) and (20-R).

The term “ginsenosides isolated from the extract of processed ginseng ” disclosed herein comprise at least one saponin or their combination selected from the group consisting of ginsenoside Rk1 of following chemical formula (1), ginsenoside Rg5 of following chemical formula (2), 20(S)-ginsenoside Rg3 of following chemical formula (3), and 20(R)-ginsenoside Rg3 of following chemical formula (4), especially, ginsenoside Rk1.

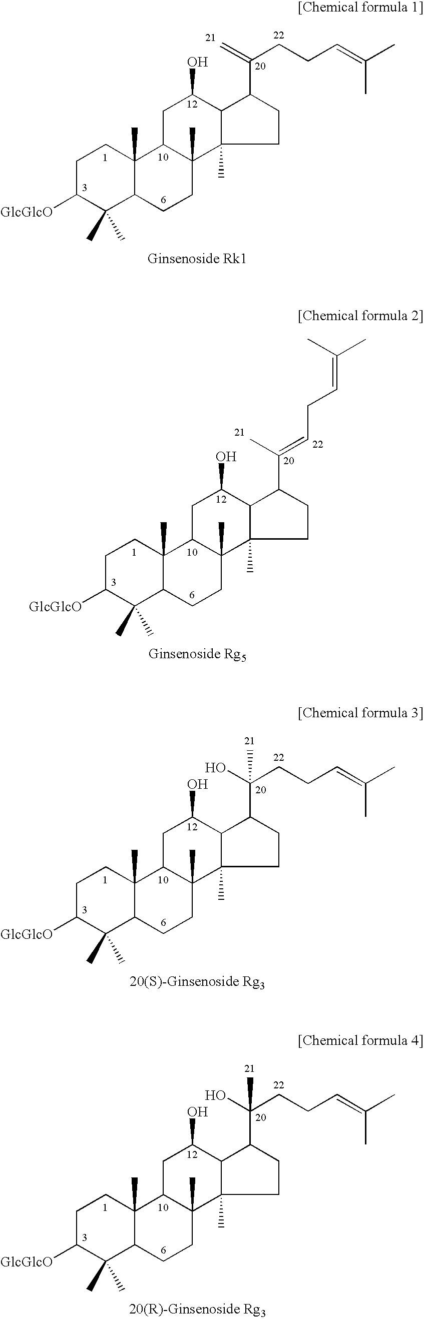

The term “Panax genus” disclosed herein comprises the root, stem, petal, leaf or fruit of Panax ginseng, Panax quinquefolia, Panax notoginseng, Panax trifolia, Panax japonica, Panax pseudoginseng, Panax vietnamensis, Panax elegatior, Panax wangianus or Panax bipinratifidus.

The term “thrombotic diseases” disclosed herein comprises the thrombotic diseases caused by platelet aggregation induced by arachidonic acid, thromboxane A2 or collagen and the thrombotic diseases caused by intrinsic factors among coagulation factors present in plasma; specifically, arteriosclerosis, embolus, ischemic cardiac disease, stroke, angina, cerebral infarction, intracranial hemorrhage, aneurysm, atherosclerosis, atheroembolus, nephrosclerosis, or cardiac infarction, more specifically, stroke, cerebral infarction, or atheroembolus.

The term “intracranial hemorrhage” disclosed herein comprises spontaneous intracranial hemorrhage or subdural hemorrhage.

The term “aneurysm” disclosed herein comprise abdominal aortic aneurysm.

Above-described extract of processed ginseng and ginsenosides isolated therefrom can be prepared in accordance with the following preferred embodiment.

Hereinafter, the present invention is described in detail.

For the present invention, for example, dried plant material of Panax genus, for examples, the root of Panax ginseng is cut into small pieces and the piece was heated at the temperature ranging from 70 to 150° C., preferably from 100 to 130° C., for the period ranging from 2 to 6, preferably 3 to 5 hrs; and was mixed with 1 to 20-fold, preferably, 3 to 10-fold weight of water, C₁-C₄ lower alcohol such as methanol, ethanol, butanol, or the mixtures thereof, preferably ethanol; and was heated for the period ranging from 3 to 10 hours, preferably 3 to 6 hours, by reflux extraction with water, cold water extraction, ultra-sonication or conventional extraction, preferably by reflux extraction with water; the residue was filtered and then the filtrate was dried at the temperature ranging from 40 to 80, preferably from 50 to 70, to obtain inventive extract of processed ginseng.

To obtain purposed ginsenosides from the inventive extract of processed ginseng, following procedure could be performed:

The inventive extract of processed ginseng prepared by above step, is suspended with water, and then is extracted with 1 to 100-fold, preferably, 1 to 5-fold volume of non-polar organic solvent such as hexane, ether, dichloromethane, chloroform, ethyl acetate or the mixtures thereof; the water-soluble layer obtained the above step is further extracted with polar organic solvent such as butanol; and the above organic solvent-soluble extract is further subjected to chromatography for increasing the content of ginsenoside by repeating the above extraction steps. The purposed ginsenosides of the present invention, i.e., ginsenoside Rg3, Rg5 or Rk1 can be obtained by repeated crystallization steps with appropriate solvent such as water, lower alcohol, lower ketone, chloroform or the mixture thereof.

Through the above described procedure, the saponins such as ginsenoside Rb₁, Rb₂, Rc, Rd etc being contained in plant material is transformed into chemically modified ginsenosides such as ginsenoside Rg₃, Rg₅, Rk₁ etc due to heat treatment.

In particular, the extract of processed ginseng according to the present invention wherein a ratio of ginsenoside (Rg3+Rg5+Rk1) to (Rb1+Rb1+Rc+Rd) of over 1.0 showing more potent physiological activities than those of extract of conventional preparations of ginseng. The extract of the present invention prepared by the above-described method may comprise pharmacologically active ginsenosides, i.e, ginsenoside Rk1, ginsenoside Rg5, 20(S)-ginsenoside Rg3 or 20(R)-ginsenoside Rg3 in the content ranging from 0.002 to 20.000% (w/w), preferably, 0.002 to 15.000% (w/w), more preferably, 0.002 to 10.000% (w/w) based on the dried weight of inventive ginseng extract.

In order to perform purposed use of the present invention, the use of the present invention may be embodied by following inventive composition. The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 0.1 to 100 mg/kg, preferably, 1 to 50 mg/kg by weight/day of the inventive extract or compounds of the present invention. The dose may be administered in single or divided into several times per day. In terms of composition, the complex herbal composition should be present between 0.01 to 95% by weight, preferably 1 to 80% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

The present inventors demonstrated that the inventive extract of the present invention and the ginsenosides isolated therefrom showed more potent activity of thrombotic disease than that of the extract of ginseng prepared by conventional method or simple processed method, by accomplishing in vitro and in vivo experiment, e.g., inhibitory effect on platelet aggregation and plasma coagulation test etc., therefore, it has been confirmed that above described inventive composition is very useful in the prevention or treatment of thrombotic disease.

In particular, the composition of the present invention more potent preventing effect on platelet aggregation and plasma coagulation than conventionally available anti-thrombotic agent, i.e., aspirin, thus it is very useful for patients susceptible with thrombotic disease.

Accordingly, it is another object of the present invention to provide a health care food comprising above described extract or ginsenosides isolated therefrom prepared by above processes and a sitologically acceptable additive to prevent or alleviate thrombotic disease.

The present invention also provides a health care food comprising the extract of processed ginseng obtained by the steps; heating Panax plant with heat at the temperature ranging from 70 to 150° C. for the period ranging from 2 to 6, and a sitologically acceptable additive to prevent or alleviate thrombotic disease

The present invention also provides a health care food comprising saponin compounds at least one or their combination selected from the group consisting of ginsenoside Rg3, Rg5 and Rk1 and a sitologically acceptable additive to prevent or alleviate thrombotic disease.

The term “a health care food” defined herein is “the food containing inventive extract of the present invention showing no specific intended effect but general intended effect in a small amount of quantity as a form of additive or in a whole amount of quantity as a form of capsule, pill, tablet etc”.

The term “a sitologically acceptable additive” defined herein is “any substance the intended use which results or may reasonably be expected to result-directly or indirectly-in its becoming a component or otherwise affecting the characteristics of any food” for example, thickening agent, maturing agent, bleaching agent, sequesterants, humectant, anticaking agent, clarifying agents, curing agent, emulsifier, stabilizer, thickner, bases and acid, foaming agents, nutrients, coloring agent, flavoring agent, sweetner, preservative agent, antioxidant, etc, which had been well-known in the art.

For example, the health care food of present invention can be prepared and processed into various forms such as tablet, capsule, powder, granule, liquid, pill etc for the purpose to alleviate and prevent thrombotic disease.

More specifically, the tablet form can be prepared by formulating the extract itself or mixed with conventional carrier, adjuvants, diluents or the other additive thoroughly into granule form and the granule is further mixed with the other additive such as lubricant etc to be compressed in order to formulate into the tablet form of health care food. In an alternative embodiment, the tablet form of health care food can be further mixed with conventional carrier, adjuvants, diluents or the other additive thoroughly to make into powder form and humidified with appropriate solvent in order to be formulated into purposed form with drying, if necessary, mixed with taste acids such as crystalline cellulose, lactose etc or coating agent such as PEG.

The capsule form can be prepared by packing the granule itself or the granule mixed with conventional carrier, adjuvants, diluents or the other additive into conventional capsule made by appropriate base such as gelatin, and plasticizer such as glycerin or sorbitol, if necessary, with coloring agent or preservative in order to be formulated into purposed form of inventive health care food.

The pill form can be prepared by formulating the granule extract itself or the extract mixed with conventional carrier, adjuvants, diluents or the other additive into conventional granule, if necessary adding with appropriate coating agent such as sucrose, starch, talc etc in order to be formulated into purposed form of inventive health care food.

The granule form can be prepared by formulating the extract itself or the granule mixed with conventional carrier, adjuvants, diluents or the other additive into conventional granule, if necessary, mixed with taste acids such as crystalline cellulose, lactose etc or coating agent such as PEG. It is preferable to permit passing through No. 12 sieve (1680 μm) with total; No. 14 sieve (1410 μm) with remaining ratio of less than 5.0% and No. 45 sieve (350 μm) with remaining ratio of less than 15.0%.

If a substance is added to a food for a specific purpose in that food, it is referred to as a direct additive and indirect food additives are those that become part of the food in trace amounts due to its packaging, storage or other handling.

Above described health foods can be contained in food, health beverage, dietary therapy etc, and may be used as a form of powder, granule, tablet, chewing tablet, capsule, beverage etc for preventing or improving aimed disease.

Above-described composition therein can be added to food, additive or beverage for prevention of thrombotic disease. For the purpose of preventing thrombotic disease, wherein, the amount of above described extract or compound in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described extract or compounds as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Examples of edible food comprising aforementioned extract or compounds therein are various food, beverage, gum, vitamin complex, health improving food and the like.

Inventive extract or compound of the present invention has no toxicity and adverse effect therefore they can be used with safe.

The present invention is more specifically explained by the following examples. However, it should be understood that the present invention is not limited to these examples in any manner.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Example 1

### Preparation of the Extract of Processed Ginseng

Specifically processed ginseng was prepared in accordance with the procedure disclosed in the literature (Kim W Y et al., J. Nat. Prod., 63(12), pp. 1702-1704; Kwon S W et al., J. Chromatogr A., 921(2), pp. 335-339, 2001).

1 kg of dried plant material of Panax genus, for examples, the root of Panax ginseng was cut into small pieces and the sliced piece was heated at 120° C. for 3 hours in autoclave. The processed ginseng was mixed with 2 liters of ethanol and heated for 4 hours by reflux extraction with water. The residue was filtered and then the filtrate was evaporated to obtain 300 g of inventive extract of processed ginseng (yield; about 30%), which designated as ‘SG’ hereinafter.

### Example 2

### Preparation of Ginsenoside Rg3, Rg5 and Rk1 Isolated from the Extract of Processed Ginseng

100 g of processed ginseng extract prepared by the above Example 1 was mixed with 1000 ml of water, and then was extracted with 1000 ml of saturate butanol with 3 times. The butanol soluble extract was further subjected to silica gel column chromatography and eluted with a ethylacetate:methanol:water mixture (20:1:1) to obtain 6 g of purified fraction containing ginsenoside Rk1 and Rg5 (designated as ‘SGBu-1’ hereinafter) by repeating the above chromatography procedure. Similarly, 5 g of the purified fraction containing racemic mixture of 20(S)-ginsenosides Rg3 and 20(R)-ginsenosides Rg3 (designated as ‘SGBu-2’ hereinafter) was obtained. The fractions were further purified over semi-preparative HPLC (two L-7100 pumps, Hitachi, Japan) coupled with evaporative light scattering detector (ELSD; Sedex, Sedere, France) using reverse phase column (C18) with eluent system (Acetonitrile/water) repeatedly. Finally, 150 mg of ginsenoside Rk1, 200 mg of ginsenoside Rg5, 200 mg of 20(S)-ginsenosides Rg3 and 100 mg of 20(R)-ginsenosides Rg3 were obtained respectively.

### Experimental Example 1

### Antiplatelet Aggregatory Activity Test

The inhibitory effect of the ginsenosides prepared in Examples on the rat platelet aggregation was determined according to the procedure disclosed in the literature (Yun-Choi et al., Modified smear method for screening potential inhibitors of platelet aggregation from plant sources, J. Nat. Prod., 48(3), pp 363-370, 1985).

### 1-1. Preparation of PRP(Platelet Rich Plasma)

The blood of 7 weeks-year old Sprgue-Dawley rat (male, 240±20 g, Orient Bio Co. Ltd) was collected using a syringe containing 2.2% sodium citrate (10% v/v) and centrifuged for 10 mins with the speed of 200× g to collect platelet rich plasma containing supernatant(PRP). 0.16 ml of PRP was treated with 0.2 ml of SG, SGbu-1, and SGBu-2 as test samples and 0.2 ml of solutions of arachidonic acid (Sigma, A8798), ADP (Sigma, A4386) and collagen (Chrono-Log, No. 385) as platelet aggregation inducers (control group) to incubate for 4 mins at 37° C.

### 1-2. Modified Smear Method

Thin Smear prepared from step 1-1 was stained and the intensity of platelet aggregation result was observed by microscopy method. The intensity of platelet aggregation result was divided into five categories, i.e., “++”: full aggregation of platelet; “+”: intermediate aggregation of platelet; “±”: slight aggregation of platelet; “−”: no aggregation of platelet.

At the result, the groups treated with SGBu-1 and SGBu-2 showed “±˜+” activities as can be seen in Table 1 at the smear test treated with arachidonic acid. Accordingly, the inhibitory effect of ginsenoside Rk1, ginsenoside Rg5, 20(S)-ginsenosides Rg3 and 20(R)-ginsenosides Rg3 on platelet aggregation was determined and the result was shown in Table 1.

TABLE 1
The inhibitory activity of ginsenosides on platelet
aggregation in modified smear method.

| | | | Aggregation inducer | | |
|---|---|---|---|---|---|
| sample | | Conc. (mg/ml) | ADP | collagen | AA* |
| Control** | PRP(−)^{a)} | — | − | − | − |
| | PRP(+)^{b)} | — | ++ | ++ | ++ |
| | PRP(AS) | — | −(±)^{c)} | −(±)^{d)} | −(±)^{e)} |
| SG | | 1.0 | ++ | +~++ | ± |
| SGBu-1 | | 1.0 | ++ | +~++ | ± |
| SGBu-2 | | 1.0 | ++ | ++ | ±~+ |

*AA: Arachidonic Acid
**^{a)}PRP which was not treated with aggregation inducer
^{b)}PRP which was treated with aggregation inducer (ADP; 3.2 × 10⁻⁶ M; collagen; 2 × 10⁻⁵ g/ml; AA; 4 × 10⁻⁴ M);
^{c)}PRP treated with 2.8 × 10⁻³ M Aspirin (5.5 × 10⁻³ M)
^{d)}PRP treated with 1.4 × 10⁻³ M Aspirin (2.8 × 10⁻³ M)
^{e)}PRP treated with 0.7 × 10⁻³ M Aspirin (1.4 × 10⁻³ M)
The test was repeated with three times

### Experimental Example 2

### Platelet Aggregation Inhibition Test

### 2-1. Preparation of Adjusted PRP(Platelet Rich Plasma)

7 weeks-year old Sprgue-Dawley male white rat (240±20 g, Orient Bio Co. Ltd) was anesthetized with diethyl ether. The blood of rat was collected from heart using a syringe containing 2.2% sodium citrate (10% v/v) and centrifuged for 10 mins with the speed of 200×g and the supernatant (upper layer) of buffy coat was collected using by serum separator (Mediland Co. Ltd, 10×250 mm) to afford platelet rich plasma containing supematant(PRP). The residue was centrifuged at 1500×g for 15 min to obtain the platelet poor plasma (PPP) and the PRP was diluted with PPP to adjust the final platelet number in PRP to 4.0-4.5×10⁸/ml. Platelet number was counted using a platelet counter (Excell 18 MWI, DANAM Electronics, USA) disclosed in the literature (Lee Y. Y. et al., Anti-platelet Aggregating and anti-oxidative activities of 11-O-(4′-O-Methylgalloy)-bergenin, A new compound isolated from Crassul cv. “Himaturi”, Planta Med., 71, pp 776-777).

### 2-2. Platelet Aggregation Inhibition Test

An aliquote (500 μl) of platelet count adjusted PRP was preincubated for 3 minutes at 37° C. in an aggregometer (490-X optical aggregometer, Chrono-Log Corp, USA) with constant stirring at 1,000 rpm. 5 μl of ginsenoside solution was added followed by successive addition of aggregation inducing agent (5 μl) at an intervals of 30 s to incubate for 30 sec with stirring at the speed of 1100 rpm. Arachidonic acid (AA, Sigma, A8798), Thromboxane A2 (U46619, Sigma, D8174) and collagen (Chrono-Log, No. 385) were used as aggregation inducing agents at the concentration of 30˜60×10⁻⁶M (AA), 2˜4×10⁻⁶M (U46619) and 2˜4×10⁻⁶ g/ml (collagen) respectively. Aspirin was used as a positive control. Because of poor solubility of ginsenosides in saline, DMSO was used as a vehicle at a concentration (1.0%) which does not affect platelet aggregation. The aggregation induced by AA and U46619 was determined in presence of a near-threshold concentration (1.0-1.4 μg/ml and 0.8-1.2 μg/ml, respectively) of collagen that can only induce change in platelet shape but no aggregation.

Inhibition (%) was calculated as follows: Inhibition (%)=(1−maximal aggregation of sample-treated aPRP/maximal aggregation of vehicle-treated aPRP)×100 All experiments were performed in tripticates. IC₅₀ values were calculated from the linear regression of the plot of mean values (n=3) of percent inhibition versus concentration of corresponding ginsenoside. Sigma Plot software was used for graphing. Regression equations were calculated using Regression Wizard from the Sigma Plot equation library.

As a result, most of the tested compounds showed dose dependent inhibitory effect to collagen, AA and U46619 (thromboxane A₂ mimetic drug)-induced platelet aggregation (Table 2). Ginsenoside Rk₁ exhibited the strongest inhibitory effect among four tested compounds on collagen, AA and U46619-induced platelet aggregation. In particular, it showed 22 fold stronger activity than ASA on AA-induced aggregation. Ginsenoside Rg₅ also showed 8 fold stronger inhibitory activity against AA-induced aggregation than ASA. 20(S)-Ginsenoside Rg₃ showed similar effect like ASA on the inhibition of AA-induced platelet aggregation, whereas, 20(R)-Rg₃ showed only poor platelet antiaggregating activity. While ginsenoside Rk₁ exhibited 3 fold higher activity than ASA on collagen-induced aggregation, activities of ginsenosides Rg₅ and 20(R)-Rg₃ were found comparable to that of ASA. Ginsenosides Rk₁ and Rg₅ showed 5-6 times strong inhibition than ASA on U46619-induced aggregation. Racemic ginsenoside Rg₃ which has deglycosylated structure at C₂₀ exhibited inhibitory effect on AA-induced platelet. In the present study, 20(S)-ginsenoside Rg₃ and 20(R)-ginsenoside Rg_{3,} the isomers of ginsenoside Rg₃ were isolated and their antiplatelet activity was examined. Interestingly, they seemed to have different activity in stereo-specific manner. 20(S)-Ginsenoside Rg₃ showed relatively strong inhibitory effect on AA-induced aggregation, while 20(R)-ginsenoside Rg₃ inhibited collagen and U46619-induced aggregation.

TABLE 2
The inhibitory effect of ginsenosides on platelet aggregation
induced by AA, U46619 and collagen

| | IC₅₀ (μM) | | |
|---|---|---|---|
| sample | AA | U46619 | collagen |
| ginsenoside Rk1 | 3 | 78 | 197 |
| ginsenoside Rg5 | 8 | 102 | 409 |
| 20(S)-ginsenosides | 53 | >700 | >700 |
| Rg3 | | | |
| 20(R)-ginsenosides | >100 | 357 | 492 |
| Rg3 | | | |
| Aspirin(ASP) | 66 | 508 | 588 |

### Experimental Example 3

### Anti-Coagulation Activity of Ginsenosides

The inhibitory effect of the ginsenosides prepared in Examples on human plasma coagulation was determined by aPTT (activated partial thromboplastin time), PT(prothrombin time) and TT(thrombin time) tests.

### 3-1. Preparation of Fresh Plasma

Fresh freeze plasma was procured from the blood bank of Seoul National University Hospital (www.snuh.org) to use in the Anti-coagulation Test in human plasma.

### 3-2. Anti-Coagulation Test

To determine aPTT, 0.1 ml plasma treated with test samples was incubated at 37° C. for 2 mins and aPTT reagent (Diagnostica Stago, Cat. 0595) was added thereto to incubate for 3 mins. The coagulation time from the starting time of plasma coagulation with 0.1 ml of 20 mM calcium chloride solution to the ending time of plasma agglutination was determined using by fibrometer (BBL Fibrosystems, Fischer Scientific, USA).

To determine PT and TT, 0.1 ml plasma treated with test samples in the case of PT and 0.2 ml plasma treated with test samples in the case of TT was incubated at 37° C. for 2 mins respectively. 2 ml of PT reagent (Diagnostica Stago, Cat. 0605) and TT reagent (Diagnostica Stago, Cat. 0611) were added thereto to incubate for 3 mins. The coagulation time from the starting time of plasma coagulation with 0.1 ml of 20 mM calcium chloride solution to the ending time of plasma agglutination was determined using by fibrometer (BBL Fibrosystems, Fischer Scientific, USA).

The group treated with aspirin was used as a positive control group.

At the result, the groups treated with ginsenoside Rk1, ginsenoside Rg5, and 20(S)-ginsenosides Rg3 showed comparable anti-coagulation activity (54.5 sec, 66.0 sec and 57.2 sec respectively) to control group (45.4 sec) as can be seen in Table 3 in aPTT test.

The groups treated with ginsenoside Rk1, and ginsenoside Rg5 also showed comparable anti-coagulation activity (AT: 29.8 sec, and 28.1 sec respectively) to control group (20.5 sec) as can be seen in Table 3 in PT test.

In conclusion, ginsenoside Rk1, and ginsenoside Rg5 showed prolonged time to plasma coagulation as well as comparable antiplatelet aggregatory activity to ASA.

TABLE 3
The inhibitory effect on human plasma agglutination

| sample | aPTT (sec) | PT (sec) | TT (sec) |
|---|---|---|---|
| ginsenoside Rk1 | 54.5 | 29.8 | 14.3 |
| ginsenoside Rg5 | 66.0 | 28.1 | 14.1 |
| 20(S)-ginsenosides Rg3 | 57.2 | 23.5 | 12.9 |
| 20(R)-ginsenosides Rg3 | 50.3 | 24.1 | 13.6 |
| Control | 45.4 | 20.5 | 15.3 |

Hereinafter, the formulating methods and kinds of excipients will be described, but the present invention is not limited to them. The representative preparation examples were described as follows.

Preparation of injection

| | |
|---|---|
| ginsenoside Rk1 | 100 mg |
| Sodium metabisulfite | 3.0 mg |
| Methyl paraben | 0.8 mg |
| Propyl paraben | 0.1 mg |
| Distilled water for injection | optimum amount |

Injection preparation was prepared by dissolving active component, controlling pH to about 7.5 and then filling all the components in 2 ml ample and sterilizing by conventional injection preparation method.

Preparation of powder

| | |
|---|---|
| ginsenoside Rk1 | 500 mg |
| Corn Starch | 100 mg |
| Lactose | 100 mg |
| Talc | 10 mg |

Powder preparation was prepared by mixing above components and filling sealed package.

Preparation of tablet

| | |
|---|---|
| ginsenoside Rk1 | 200 mg |
| Corn Starch | 100 mg |
| Lactose | 100 mg |
| Magnesium stearate | optimum amount |

Tablet preparation was prepared by mixing above components and entabletting.

Preparation of capsule

| | |
|---|---|
| ginsenoside Rk1 | 100 mg |
| Lactose | 50 mg |
| Corn starch | 50 mg |
| Talc | 2 mg |
| Magnesium stearate | optimum amount |

Tablet preparation was prepared by mixing above components and filling gelatin capsule by conventional gelatin preparation method.

Preparation of liquid

| | | |
|---|---|---|
| Ginsenoside Rk1 | 1000 | mg |
| Sugar | 20 | g |
| Polysaccharide | 20 | g |
| Lemon flavor | 20 | g |

Liquid preparation was prepared by dissolving active component, and then filling all the components in 1000 ml ample and sterilizing by conventional liquid preparation method.

Preparation of health food

| ginsenoside Rk1 | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B6 | 0.5 | mg |
| Vitamin B12 | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above mentioned vitamin and mineral mixture may be varied in many ways. Such variations are not to be regarded as a departure from the spirit and scope of the present invention.

Preparation of health beverage

| | | |
|---|---|---|
| ginsenoside Rk1 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 2000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the extract of processed Panax genus plant and saponin compounds isolated therefrom show present invention more potent preventing effect on platelet aggregation and plasma coagulation than conventionally available anti-thrombotic agent, i.e., aspirin, thus it is very useful for patients susceptible with thrombotic disease.

1. A method for treating or preventing thrombotic diseases in mammals, wherein the method comprises administering a therapeutically effective amount of an extract of processed ginseng or ginsenosides isolated therefrom into the mammal suffering with thrombotic diseases.

2. The method according to claim 1, said extract of processed ginseng is prepared by the steps; treating Panax genus plants with heat at the temperature ranging from 70 to 150° C. for the period ranging from 2 to 6, as an active ingredient in an amount effective to treat or prevent thrombotic disease.

3. The method according to claim 1, said ginsenosides is at least one ginsenoside or their combination selected from the group consisting of ginsenoside Rk1, ginsenoside Rg5, 20(S)-ginsenoside Rg3, and 20(R)-ginsenoside Rg3.

4. The method according to claim 1, said thrombotic diseases is arteriosclerosis, embolus, ischemic cardiac disease, stroke, angina, cerebral infarction, intracranial hemorrhage, aneurysm, atherosclerosis, atheroembolus, nephrosclerosis, or cardiac infarction.

5. A pharmaceutical composition comprising an extract of processed ginseng and ginsenosides isolated therefrom as set forth in claim 1 as an active ingredient in an effective amount to treat and prevent thrombotic diseases.

6. A functional health food comprising an extract of processed ginseng or ginsenosides isolated therefrom as set forth in claim 1 together with a sitologically acceptable additive for the prevention and improvement of thrombotic diseases.
